(19) United States
(12) Patent Application Publication
(10) Pub. No.: US 2024/0342175 A1
(43) Pub. Date: Oct. 17, 2024

(54) Compositions and Methods for Treating Spinal Cord Injury and Synaptic Dysfunction

(71) Applicant: Howard Mitz, Littleton, NH (US)

(72) Inventor: Howard Mitz, Littleton, NH (US)

(21) Appl. No.: 18/617,800

(22) Filed: Mar. 27, 2024

Related U.S. Application Data
(63) Continuation of application No. 17/711,247, filed on Apr. 1, 2022, now abandoned.
(60) Provisional application No. 63/170,870, filed on Apr. 5, 2021.

(51) Int. Cl. A61K 31/517; A61K 31/16; A61K 31/167; A61K 31/192; A61K 31/22; A61K 31/352; A61K 31/381; A61K 31/40; A61K 31/401; A61K 31/428; A61K 31/437; A61K 31/44; A61K 31/4409; A61K 31/4439; A61K 31/472; A61K 31/506; A61K 31/5377; A61K 31/541; A61K 31/551; A61K 31/65; A61K 35/60; A61K 36/00; A61K 38/57; A61P 25/00

(52) U.S. Cl. CPC A61K 31/517; A61K 31/16; A61K 31/167; A61K 31/192; A61K 31/22; A61K 31/352; A61K 31/381; A61K 31/40; A61K 31/401; A61K 31/428; A61K 31/437; A61K 31/44; A61K 31/4409; A61K 31/4439; A61K 31/472; A61K 31/506; A61K 31/5377; A61K 31/541; A61K 31/551; A61K 31/65; A61K 35/60; A61K 36/00; A61K 38/57; A61P 25/00

(57) ABSTRACT

The present invention provides methods and compositions for treating synaptic dysfunction and spinal cord injuries comprising, for example, the administration of a compound that increases the level of PZP in vivo.

### RELATED APPLICATIONS

This application is a continuation of U.S. application Ser. No. 17/711,247, filed on Apr. 1, 2022, which claims the benefit of U.S. Provisional Application No. 63/170,870, filed on Apr. 5, 2021. The entire teachings of the above applications are incorporated herein by reference.

### BACKGROUND OF THE INVENTION

Pregnancy zone protein (PZP) was first discovered in 1959 by Smithies (Smithies, O Adv. Protein Chemistry 1959 14, 68-118). The gene for PZP is found on chromosome 12. Its location is 12 P 13-P 12.2 (Pregnancy zone protein OS=Homo sapiens GN=PZP PE=1 SV=4, Accession #sp|P20742|PZP_HUMAN and GenBank: X54380.1). PZP, a homotetramer, uses a mechanism of trapping that can inhibit all four classes of proteinases. This protein contains cleavage sites for several proteinases. Upon binding of a proteinase, the conformation of this protein changes to trap the proteinase, limiting its activity. Levels of PZP have been found in tissues including the brain. It has also been found in serum and plasma. It has been hypothesized that PZP, in concert with another protein, may modulate T cell activation. PZP is normally a trace protein that is strongly upregulated during pregnancy. However, the purpose of PZP in the non-pregnant state is still not known.

Recently, PZP has been correlated to Intellectual Disability (ID). Mitz, Howard, WO2017/062238, published Apr. 13, 2017, which is incorporated herein by reference in its entirety. Specifically, elevated levels of PZP were found in patients diagnosed with Downs syndrome and Alzheimer's Disease, while decreased levels of PZP was found in patients diagnosed with Fragile X syndrome, Koolen-DeVries and syndrome and Niemann-Pick disease, for example. Mitz teaches that ID, such as that correlated with these conditions, can be treated by normalizing PZP levels.

### SUMMARY OF THE INVENTION

The invention is based, in part, on the discoveries that (1) PZP, and related proteins, can improve or treat synaptic dysfunction and, thereby, treat impaired or severed nerves, such as can be found in patients suffering from a spinal cord injury and (2) ROCK (or more, specifically ROCK2) inhibitors and other compounds increase the expression of PZP. Thus, the invention provides compositions and methods of treating or ameliorating synaptic dysfunction in a subject, preferably a human patient.

Such compositions may be pharmaceutical compositions comprising at least one therapeutic agent capable of increasing the levels or activity of PZP in a subject, preferably a human subject, and an optional pharmaceutically acceptable carrier.

### DETAILED DESCRIPTION OF THE INVENTION

The present invention is based, in part, on results which demonstrate that underexpression or overexpression of human Pregnancy Zone Protein (hPZP) is associated with intellectual disability (ID) resulting from disparate causes of ID, as described in Mitz, Howard, WO2017/062238, published Apr. 13, 2017, which is incorporated herein by reference in its entirety. A subject with ID generally has lower than average intelligence. Intelligence describes a subject's ability to think, learn and solve problems. ID is commonly classified according to Intelligent Quotient (IQ). The Diagnostic & Statistical Manual of Mental Disorders (4th Edition; DSM-IV, 1994) identifies mild MR in the IQ range 50-55 to 70, moderate MR as 35-40 to 55-55, severe MR as 20-25 to 35-40, and profound MR as below 20-25. A subject with ID may have difficulty learning, may take longer to learn social skills, such as how to communication and may be less able to care for himself or herself and to live on his or her own as an adult.

One aspect of the invention relates to the appreciation that ROCK inhibitors increase the expression of PZP. ROCK refers to the Rho kinase signaling pathway, which has been described for neurodegenerative disorders, such as Parkinson's disease, Alzheimer's disease and amyotrophic lateral sclerosis. ROCK2 refers to the isoform expressed in the brain, also called ROKα. The human isoforms share significant sequence identity (about 65%) with a highly conserved sequence in the catalytic domain (about 92%). Koch et al., ROCK inhibition in models of neurodegeneration and its potential for clinical translation, Pharmacology & Therapeutics, 189 (2018) 1-21, which is incorporated herein by reference.

A preferred ROCK inhibitor that can be used in the invention include belumosudil, which has selectivity for ROCK2. Other ROCK inhibitors include, for example, fasudil, hydroxyfasudil, ripasudil, netarsudil, RKI-1447, Y-27632, GSK429286A, Y-30141, GSK429286A, GSK180736A, GSK269962A, Thiazovivin, AT13148, H1152, Glycyl-H1152, TC-S 7001, AS-1892802, HA-1100, OXA-06, SB-772077B, SR-3677, KD025 (SLx-2119), Y-30141, Y-39983, or ZINC00881524.

ROCK1/2 pan/inhibitors include ripasudil, RKI-1447, Y-27632, GSK429286A, Y-30141, thiazovivin, GSK180736A, GSK269962A, netarsudil, Y-39983, ZInC00881524, Yf-356{(+)-(R)-4-(1-Aminoethyl)-N-(4-pyridyl) benzamide}, Rho kinase inhibitor IV {(S)-(+)-2-Methyl-4-glycyl-1-(4-methylisoquinolinyl-5-sulfonyl) homopiperazine, H-1152}, Rho kinase inhibitor II {N-(4-Pyridyl)-N′-(2,4,6-trichlorophenyl)urea}, SB772077B, Rho kinase inhibitor III {(3-(4-Pyridyl)-1H-indole)}, K-115, HA1100, rhostatin, CCG-1423 {N-(2-(4-Chloroanilino)-1-methyl-2-oxoethoxy)-3,5-bis(tri-fluoro-methyl)benzamide}, cethrin (VX-210), BA-210, BA-1042, BA-1043, BA-1044, BA-1050, BA-1051, BA-1076, BA-215, BA-285, BA-1037, Ki-23095, and AT13148.

ROCK 2 specific inhibitors include fasudil, KD-025 {2-(3-(4-((1H-indazol-5-yl)amino)quinazolin-2-yl)phenoxy)-N-isopropylacet-amide)}, BA-1049, Rho kinase inhibitor V {N-(4-(1H-pyrazol-4-yl)phenyl)-2,3-dihydrobenzo[b][1,4]dioxine-2-carboxam-ide}, SR3677, and LYC-53976.

Belumosudil has been granted a New Drug Application a priority review designation for the treatment of chronic graft-versus-host-disease (GVHD) in November 2020.

Additional compounds or compositions have been shown to increase PZP expression, as well. For example, the Chinese herb Guzhi Zengsheng Zhitongwan, or GZZSZTW, can increase PZP. GZZSZTW can be extracted from Rehmannia glutinosa, Spatholobus suberectus, Dunn, Epimedium brevicornu Maxim, Raphanus sativus L. (baked), Drynaria fortunei (baked), Cynomorium coccineum subsp. songaricum and Cibotiumbarometz (L.). In other literature, the herb is described as comprising Shu Di (Rehmannia glutinosa (Gaertn.) DC.), Ying Yang Huo (Epimedium brevicornu Maxim (K. S. Hao)), Gu Sui Bu (Drynaria fortunei (Kunze ex Mett.) J.Sm. (baked)), Suo Yang (Cynomorium coccineum subsp. songaricum (Rupr.) (J. Léonard)), and Gou Ji (Cibotium barometz (L.) (J.Sm)) and can be obtained from the Hospital of Changchun University of Chinese Medicine (Changchun, China). It is likely that one or more components of the traditional medicine is inert to increasing PZP levels. Each combination and permutation of components, eliminating inert components is intended as if listed separately. Each combination can then be screened to identify the PZP active components.

Additionally, matrix metalloproteinase 9 may decrease PZP levels in vivo. Accordingly administering an MMP-9 inhibitor may also increase PZP levels. MMP-9 inhibitors include Actinonin, which is also known as Butanediamide, N4-hydroxy-N1-[(1S)-1-[[(2S)-2-(hydroxymethyl)-1-pyrrolidinyl]carbonyl]-2-methylpropyl]-2-pentyl-, (2R)-(9C1); epigallocatechin gallate; collagen peptidomimetic and non-peptidomimetic inhibitors; tetracycline derivatives, e.g., hydroxamate peptidomimetic inhibitor batimastat; and its orally-bioavailable analogue marimastat, prinomastat, metastat, Neovastat, Tanomastat, TAA211, MM1270B or AAJ996. The MMP-9 inhibitor can also be selected from the group doxycycline, minocycline, pravastatin, captopril and a beta blocker. The MMP-9 inhibitor is preferably minocycline.

In addition, PZP interacts with brain-derived neurotrophic factor binding and nerve growth factor binding.

The compositions of the invention can include the administration of one or more, preferably two or more, of the compounds described above in an amount therapeutically effective to increase the levels of PZP expression. In increasing PZP expression, the composition can treat or restore synaptic dysfunction.

Synaptic dysfunction can arise from a trauma, injury or iatrogenic event to a nerve, such as a peripheral nerve, spinal cord, brain or cranial nerves. Regeneration of nerves and restoration of function synapses is a significant unmet need. In the context of treating such conditions, the methods of the invention include enhancing nerve growth, regrowth or regeneration, and restoring synapse function. In other embodiments, the synaptic dysfunction can arise from a congenital disorder, autoimmune disease or iatrogenic event. The spinal nerve injury can be cervical, lumbosacral or thoracic. The method may result in improved motor control in said subject, such as fine motor control, gross motor control or autonomic nerve control.

Nerve injury, broadly defined, is injury to nervous tissue. There is no single classification system that can describe all the many variations of nerve injury. Nerve injuries can include injuries to the nerve fiber and discontinuity. Nerve injury can incur damage to both the nerve and the surrounding connective tissue, since supporting glial cells may be involved. The processes that occur in peripheral regeneration can be divided into the following major events: Wallerian degeneration, axon regeneration/growth, and end-organ reinnervation. The events that occur in peripheral regeneration occur with respect to the axis of the nerve injury. The proximal stump refers to the end of the injured neuron that is still attached to the neuron cell body; it is the part that regenerates. The distal stump refers to the end of the injured neuron that is still attached to the end of the axon; it is the part of the neuron that will degenerate but that remains in the area toward which the regenerating axon grows. The restoration of PZP levels can improve restoration of synapse function.

A spinal cord injury (SCI) or defect is an injury to the spinal cord resulting in a disruption, either temporary or permanent, in the cord's normal motor, sensory, or autonomic function. Common causes of damage are trauma (car accident, gunshot, falls, sports injuries, etc.) or disease (transverse myelitis, polio, spina bifida, Friedreich's ataxia, etc.). The spinal cord does not have to be severed in order for a loss of function to occur. Depending on where the spinal cord and nerve roots are damaged, the symptoms can vary widely, from pain to paralysis to incontinence. Spinal cord injuries are described at various levels of “incomplete,” which can vary from having no effect on the patient to a “complete” injury which means a total loss of function.

Nerve trauma can also include brain injury or cranial nerve deficits. Brain damage or brain injury (BI) is the destruction or degeneration of brain cells, including nerves. Brain injuries occur due to a wide range of internal and external factors. A common category with the greatest number of injuries is traumatic brain injury (TBI) following physical trauma or head injury from an outside source, and the term acquired brain injury (ABI) is used in appropriate circles to differentiate brain injuries occurring after birth from injury due to a disorder or congenital malady.

In general, brain damage refers to significant, undiscriminating trauma-induced damage, while neurotoxicity typically refers to selective, chemically induced neuron damage. Brain injuries occur due to a very wide range of conditions, illnesses, injuries, and as a result of iatrogenesis (adverse effects of medical treatment). Possible causes of widespread brain damage include birth hypoxia, prolonged hypoxia (shortage of oxygen), poisoning by teratogens (including alcohol), infection, and neurological illness. Chemotherapy can cause brain damage to the neural stem cells and oligodendrocyte cells that produce myelin. Common causes of focal or localized brain damage are physical trauma (traumatic brain injury, stroke, aneurysm, surgery, other neurological disorder), and poisoning from heavy metals including mercury and compounds of lead.

Cranial nerve disease is an impaired functioning of one of the twelve cranial nerves. It is possible for a disorder of more than one cranial nerve to occur at the same time, if a trauma occurs at a location where many cranial nerves run together, such as the jugular fossa. A brainstem lesion could also cause impaired functioning of multiple cranial nerves, but this condition would likely also be accompanied by distal motor impairment.

The facial nerve controls the muscles in the face. Facial nerve palsy is more abundant in older adults than in children and is said to affect 15-40 out of 100,000 people per year. This disease comes in many forms which include congenital, infectious, traumatic, neoplastic, or idiopathic. The most common cause of this cranial nerve damage is Bell's palsy (idiopathic facial palsy) which is a paralysis of the facial nerve. Although Bell's palsy is more prominent in adults it seems to be found in those younger than 20 or older than 60 years of age. Bell's palsy is thought to occur by an infection of the herpes virus which may cause demyelination and has been found in patients with facial nerve palsy. Symptoms include flattening of the forehead, sagging of the eyebrow, and difficulty closing the eye and the mouth on the side of the face that is affected. The inability to close the mouth causes problems in feeding and speech. It also causes lack of taste, lacrimation, and sialorrhea.

Examples of other conditions wherein a subject may underexpress PZP include Koolen-deVries Syndrome, Fragile X syndrome and syndrome and Niemann-Pick disease.

Fragile X syndrome is associated with a fragile site expressed as an isochromatid gap in the metaphase chromosome at map position Xq 27.3. Fragile X syndrome is a genetic disorder caused by a mutation in the 5′-untranslated region of the fragile X mental retardation 1 (FMR1) gene, located on the X chromosome. The mutation that causes fragile X syndrome is associated with a CGG repeat in the fragile X mental retardation gene FMR-1. When a subject has more than about 200 CGG repeats, the fragile X gene is hypermethylated, silenced, fragile X mental retardation protein (FMRP) is not produced and the subject is diagnosed as having fragile X syndrome. The fragile X syndrome segregates as an X-linked dominant disorder with reduced penetrance. Either sex when carrying the fragile X mutation may exhibit ID, which is variable in severity. Children and adults with fragile X syndrome have varying degrees of ID or learning disabilities and behavioral and emotional problems, including autistic-like features and tendencies. Fragile X syndrome can be diagnosed by an established genetic test performed on a sample (e.g., blood sample, buccal sample) from the subject. The test determines whether a mutation or pre-mutation is present in the FMR-1 gene of the subject. Patients with Fragile X syndrome can underexpress PZP.

Thus, the invention includes the treatment of a patient with Fragile X syndrome by administering an effective amount of a compound that increases PZP levels, wherein the compound is selected from a ROCK inhibitor, a MMP-9 inhibitor and GZZSZTW, or an active fraction thereof. Each such compound can also be administered together with PZP.

Koolen deVries syndrome, formerly known as 17q21.31 microdeletion syndrome, is a condition caused by a small deletion of genetic material from chromosome 17. The deletion occurs at a location designated as q21.31. People with 17q21.31 microdeletion syndrome may have developmental delay, intellectual disability, seizures, hypotonia, distinctive facial features, and vision problems. Some affected individuals have heart defects, kidney problems, and skeletal anomalies such as foot deformities. The exact size of the deletion varies among affected individuals, but it contains at least six genes. This deletion affects one of the two copies of chromosome 17 in each cell. The signs and symptoms of 17q21.31 microdeletion syndrome are probably related to the loss of one or more genes in this region. Patients with Koolen deVries syndrome can underexpress PZP.

Thus, the invention also includes the treatment of a patient with Koolen de Vries syndrome by administering an effective amount of a compound that increases PZP levels, wherein the compound is selected from a ROCK inhibitor, a MMP-9 inhibitor and GZZSZTW, or an active fraction thereof and combinations thereof. Each such compound can also be administered together with PZP.

Niemann-Pick Disease is an example of a disease wherein a subject may underexpress PZP. Niemann-Pick Disease refers to a group of lysosomal storage diseases that include Types A (NPA), B (NPB), C (NPC) and sometimes a Type D (NPD) that is also referred to as Type A/B. In the Type C form of Niemann-Pick Disease (NPC), patients are not able to metabolize cholesterol and other lipids properly in the cell. Consequently, excessive amounts of cholesterol accumulate in the liver and spleen and excessive amounts of other lipids accumulate in the brain causing neurological symptoms including ID, learning disabilities, and delayed development of fine motor skills. NPC is always fatal. The majority of children with NPC die before age 20. Children diagnosed with NPA also die at an early age due to severe neurological complications resulting from NPA.

NPC is caused by mutations in the NPC1 gene (NPC type 1C) or the NPC2 gene (NPC type 2C) and is inherited in an autosomal recessive manner. Investigators have determined that the NPC1 gene is located on the long arm (q) of chromosome 18 (18q11.2). The NPC2 gene is located on the long arm of chromosome 14 (14q24.3). It is believed that the underexpression of PZP in NPC, for example, is dependent on which genes are mutated.

Thus, the invention includes the treatment of a patient with Niemann-Pick disease, such as NPC, by administering an effective amount of a compound that increases PZP levels, wherein the compound is selected from a ROCK inhibitor, a MMP-9 inhibitor and GZZSZTW, or an active fraction thereof. Each such compound can also be administered together with PZP.

Again, the inventor submits that PZP can also be useful to treat synaptic dysfunction arising from injuries to the nervous system, such as severed nerves and spinal cord injuries. Thus, the invention includes the treatment of a patient with traumatic nerve injury by administering an effective amount of a compound that increases PZP levels, wherein the compound is selected from a ROCK inhibitor, a MMP-9 inhibitor, GZZSZTW, or an active fraction thereof, PZP, a vector comprising an oligonucleotide that expresses PZP and combinations thereof.

As defined herein, “normal levels of PZP” in a human subject is defined to be in the ranges of 0.02 mg/L-11 mg/L for a human male and 0.47 mg/L-77 mg/L for a human female who is not pregnant. The invention provides methods of establishing normal levels of PZP in a human subject. Such methods include, but are not limited to, administering purified or recombinant PZP to a patient that underexpresses PZP. Such methods include but are not limited to administering an agent that reduces PZP expression to a patient that overexpresses PZP.

It can be beneficial to administer a ROCK inhibitor, MMP-9 inhibitor, GZZSZTW and/or a PZP-containing composition together with a nerve growth factor or similar compound that promotes neuronal cell growth.

The term “subject” is intended to include mammals. Preferably the subject is a human subject. A human subject includes human embryos and human fetuses. The terms “subject” and “patient” may be used interchangeably herein.

“Treatment” refers to the administration of a therapeutic agent or the performance of medical procedures with respect to a patient or subject, for either prophylaxis (prevention) or to cure or reduce the symptoms of the infirmity, malady, condition or injury in the instance where the patient is afflicted. A “therapeutically effective amount” is an amount sufficient to decrease, prevent or ameliorate the symptoms associated with ID.

Compositions of the invention may be pharmaceutical compositions optionally comprising at least one pharmaceutically acceptable excipient. As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; water, pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Compositions and methods of the invention to increase levels of PZP in a subject include administering an agent that increases the level or activity of PZP in a subject in need thereof. Such compositions are called “PZP-containing compositions.” The term is intended to include pharmaceutically acceptable composition comprising an active agent selected from the group consisting of purified or recombinant PZP (UniProt P20742-PZP human) and active fragments thereof, PZP mimetics, cells expressing recombinant PZP or any active fragment thereof, a recombinant nucleic acid encoding PZP or active fragments thereof, and a vector comprising nucleic acids encoding PZP or active fragments thereof. As used herein a “vector” is a recombinant nucleic acid construct, such as a plasmid, phage genome, virus genome, cosmid or artificial chromosome, to which another DNA segment may be attached.

Preferably, a PZP-containing composition of the invention is a pharmaceutical composition comprising recombinant human PZP and a pharmaceutically acceptable excipient.

Agents of the invention may be designed such that they more readily cross the blood brain barrier (BBB). This can be desired where the synaptic dysfunction is in the brain, for example. One such system is described by Pardridge, Expert Opin. Drug Deliv. (2015) 12(2):207-222 using BBB molecular Trojan horses to deliver biologic drugs across the BBB.

For example, a BBB Trojan horse may be an endogenous peptide or peptidomimetic monoclonal antibody (Mab) that crosses the BBB via transport on an endogenous receptor-mediated transport (RMT) system. Another Trojan horse approach is described in Dietz and Bohr, Mol. Cell. Neurosci. (2004) 27(2):85-131.

Other modulators useful for opening the BBB ranging from chemical and biological substances to physical stimuli such as high frequency focused ultrasound and electromagnetic fields are also contemplated for their impact on the BBB to assist the compositions of the invention in reaching the brain, when necessary. Such stimuli/agents include, but are not limited to cyclodextrin, poloxamers, cell penetrating peptides such as MAP, Transportan, SBP, FBP, SynB1, SynB3, pAntp₄₃₋₆₈, TAT₄₈₋₆₀, viruses, Cereport (RMP-7), physical ultrasound, microwave, electromagnetic fields, nanocarriers, and PEGylated nanocarriers.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Topical administration to an injury or trauma can include application to an open wound.

It can be advantageous to administer the composition in a matrix to promote synaptic repair and neuronal cell growth. For example, a physical support structure can be placed into the critical gap of the trauma or injury, such as a structure composed of poly-lactide acid, polyurethane, polydioxanone, silicone, cellulose, collagen, PLGA, polycaprolactone or processed natural extracellular matrix. The method may further comprise administering to said subject one or more nerve growth factors, such as a neurotrophic (NGF, BDNG, NT-3), a glial-derived (GDNF) and/or a pleotropic (PTN, VEGF) nerve growth factor. Matrices, such as PLGA, can release the active components over time in a controlled and sustained fashion.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

Actual dosage levels of the pharmaceutical agents described herein may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound, the route of administration, the time of administration, the rate of excretion of the particular compound being employed, the duration of the treatment, other drugs, compounds and/or materials used in combination, the age, sex, weight, condition, general health and prior medical history of the patient being treated, and like factors well known in the medical arts.

A physician can determine and prescribe the effective amount of the pharmaceutical agent required. For example, the physician could start with doses of the compounds described herein at levels lower than that required in order to achieve the desired therapeutic effect and gradually increase the dosage until the desired effect is achieved. Accordingly, the disclosure provides a method of treating an individual, preferably a human, in need thereof comprising administering a therapeutically effective amount of a pharmaceutical agent as disclosed herein. Preferably the agent is administered intracerebrally or into the spinal cord of the individual.

With regard to belumosudil, the drug was found to be safe and efficacious in a clinical trial where 132 patients were randomized to receive belumosudil 200 mg once daily (QD) or 200 mg twice daily (BID). The AEs seen in the QD and BID arms included fatigue (30%, 18%), diarrhea (24%, 18%), nausea (23%, 20%), liver related investigations (20%, 23%), edema (24%, 15%), cough (18, 14%) dyspnea (20%, 12%), respectively. Serious AEs occurred in >2% of patients and included pneumonia (3%, 3%), nausea (3%, 2%), pyrexia (5%, 0) and vomiting (3%, 2%) in the QD and BID arms, respectively. This information can be considered by a physician to determine the dose to be administered in the present methods.

### Example 1

Fmr1 knock-out mice (n=10) were orally administered 10 mg/kg/day or 100 mg/kg/day belumosudil or vehicle (BID) for 15 days. Control groups included wild type mice and knock out mice with vehicle. Mice treated with 100 mg/kg belumosudil ameliorated the following behavioral phenotypes: open field/hyperactivity, self-grooming/stereotypy, hyponeophagia/anxiety, nesting/test of daily living, 3-chamber social novelty/sociability; resident intruder and fear conditioning. The treatment did not normalize behavior in the novel object recognition/cognition test.

The experiment was repeated where knock out mice received belumosudil at 20 mg/kg/day, 60 mg/kg/day and 100 mg/kg/day (BID) for 15 days. One group of knock out mice received 10 mg/kg/day belumosudil in combination with 10 mg/kg/day minocycline for 15 days. Control groups included wild type mice and knock out mice with vehicle. The results are set forth below, where X indicates failure to ameliorate the phenotype and Y reversed the phenotype:

| | Belumos. | Belumos. | Belumos. | Belumos with |
|---|---|---|---|---|
| Behavior | (20 mg/kg) | (60 mg/kg) | (100 mg/kg) | Minocycline |
| Open Field: Hyperactivity | X | X | Y | Y |
| Self-Grooming: Stereotypy | X | X | Y | Y |
| Hyponeophagia: Anxiety | X | X | Y | Y |
| Nesting: Test of Daily Living | Y | Y | Y | Y |
| Novel Object Recognition: Cognition | X | X | X | Y |
| 3 Chamber Social Novelty: Sociability | X | X | Y | Y |
| Resident Intruder | X | Y | Y | Y |
| Fear Conditioning | X | Y | Y | Y |

The combination of belumosudil and minocycline unexpectedly reversed all 8 phenotypes in this Fragile X mouse model.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A method of treating synaptic dysfunction in a patient in need thereof comprising administering a therapeutically effective amount of a compound selected from a ROCK inhibitor, a MMP-9 inhibitor and GZZSZTW, or an active fraction thereof, or a PZP-containing composition and combinations thereof.

2. The method of claim 1, wherein the synaptic dysfunction is a traumatic nerve injury.

3. The method of claim 1, wherein the synaptic dysfunction is a spinal cord injury.

4. The method of claim 1, wherein the compound is a ROCK inhibitor.

5. The method of claim 4, wherein the ROCK inhibitor is a ROCK2 inhibitor, preferably Belumosudil.

6. The method of claim 4, wherein the ROCK inhibitor is selected from the group consisting of ripasudil, RKI-1447, Y-27632, GSK429286A, Y-30141, thiazovivin, GSK180736A, GSK269962A, netarsudil, Y-39983, ZInC00881524, Yf-356{(+)-(R)-4-(1-Aminoethyl)-N-(4-pyridyl) benzamide}, Rho kinase inhibitor IV {(S)-(+)-2-Methyl-4-glycyl-1-(4-methylisoquinolinyl-5-sulfonyl) homopiperazine, H-1152}, Rho kinase inhibitor II {N-(4-Pyridyl)-N′-(2,4,6-trichlorophenyl)urea}, SB772077B, Rho kinase inhibitor III {(3-(4-Pyridyl)-1H-indole)}, K-115, HA1100, rhostatin, CCG-1423 {N-(2-(4-Chloroanilino)-1-methyl-2-oxoethoxy)-3,5-bis(tri-fluoro-methyl)benzamide}, cethrin (VX-210), BA-210, BA-1042, BA-1043, BA-1044, BA-1050, BA-1051, BA-1076, BA-215, BA-285, BA-1037, Ki-23095, and AT13148.

7. The method of claim 1, wherein the compound is a MMP-9 inhibitor.

8. The method of claim 7, wherein the MMP-9 inhibitor is selected from the group consisting of Actinonin, N4-hydroxy-N1-[(1S)-1-[[(2S)-2-(hydroxymethyl)-1-pyrrolidinyl]carbonyl]-2-methylpropyl]-2-pentyl-, (2R)-(9C1); epigallocatechin gallate; batimastat, marimastat, prinomastat, metastat, Neovastat, Tanomastat, TAA211, MM1270B or AAJ996.

9. The method of claim 1, wherein the composition comprises GZZSZTW.

10. The method of claim 1, wherein the patient is diagnosed with Fragile X syndrome, Koolen deVries syndrome or Niemann Pick disease.

11. The method of claim 10, wherein the compound is a ROCK inhibitor.

12. The method of claim 11, wherein the ROCK inhibitor is a ROCK2 inhibitor, preferably Belumosudil.

13. The method of claim 11, wherein the ROCK inhibitor is selected from the group consisting of ripasudil, RKI-1447, Y-27632, GSK429286A, Y-30141, thiazovivin, GSK180736A, GSK269962A, netrasudil, Y-39983, ZInC00881524, Yf-356{(+)-(R)-4-(1-Aminoethyl)-N-(4-pyridyl) benzamide}, Rho kinase inhibitor IV {(S)-(+)-2-Methyl-4-glycyl-1-(4-methylisoquinolinyl-5-sulfonyl) homopiperazine, H-1152}, Rho kinase inhibitor II {N-(4-Pyridyl)-N′-(2,4,6-trichlorophenyl)urea}, SB772077B, Rho kinase inhibitor III {(3-(4-Pyridyl)-1H-indole)}, K-115, HA1100, rhostatin, CCG-1423 {N-(2-(4-Chloroanilino)-1-methyl-2-oxoethoxy)-3,5-bis(tri-fluoro-methyl)benzamide}, cethrin (VX-210), BA-210, BA-1042, BA-1043, BA-1044, BA-1050, BA-1051, BA-1076, BA-215, BA-285, BA-1037, Ki-23095, and AT13148.

14. The method of claim 10, wherein the compound is a MMP-9 inhibitor.

15. The method of claim 14, wherein the MMP-9 inhibitor is selected from the group consisting of Actinonin, N4-hydroxy-N1-[(1S)-1-[[(2S)-2-(hydroxymethyl)-1-pyrrolidinyl]carbonyl]-2-methylpropyl]-2-pentyl-, (2R)-(9C1); epigallocatechin gallate; batimastat, marimastat, prinomastat, metastat, Neovastat, Tanomastat, TAA211, MM1270B or AAJ996.

16. The method of claim 14, wherein the MMP-9 inhibitor is selected from the group consisting of doxycycline, minocycline, pravastatin, captopril and a beta blocker.

17. The method of claim 10, wherein the ROCK inhibitor is belumosudil and the MMP-9 inhibitor is minocycline.

18. The method of claim 10, wherein the composition comprises GZZSZTW.

19. The method of claim 1, comprising administering a PZP-containing composition.

20. A pharmaceutical composition comprising an effective amount of a ROCK inhibitor and an effective amount of a MMP-9 inhibitor.

21. The composition of claim 20, wherein the ROCK inhibitor is selected from the group consisting of Belumosudil, ripasudil, RKI-1447, Y-27632, GSK429286A, Y-30141, thiazovivin, GSK180736A, GSK269962A, netarsudil, Y-39983, ZInC00881524, Yf-356{(+)-(R)-4-(1-Aminoethyl)-N-(4-pyridyl) benzamide}, Rho kinase inhibitor IV {(S)-(+)-2-Methyl-4-glycyl-1-(4-methylisoquinolinyl-5-sulfonyl) homopiperazine, H-1152}, Rho kinase inhibitor II {N-(4-Pyridyl)-N′-(2,4,6-trichlorophenyl)urea}, SB772077B, Rho kinase inhibitor III {(3-(4-Pyridyl)-1H-indole)}, K-115, HA1100, rhostatin, CCG-1423 {N-(2-(4-Chloroanilino)-1-methyl-2-oxoethoxy)-3,5-bis(tri-fluoro-methyl)benzamide}, cethrin (VX-210), BA-210, BA-1042, BA-1043, BA-1044, BA-1050, BA-1051, BA-1076, BA-215, BA-285, BA-1037, Ki-23095, and AT13148.

22. The composition of claim 20, wherein the MMP-9 inhibitor is selected from the group consisting of Actinonin, N4-hydroxy-N1-[(1S)-1-[[(2S)-2-(hydroxymethyl)-1-pyrrolidinyl]carbonyl]-2-methylpropyl]-2-pentyl-, (2R)-(9C1); epigallocatechin gallate; batimastat, marimastat, prinomastat, metastat, Neovastat, Tanomastat, TAA211, MM1270B or AAJ996.

23. The composition of claim 20, wherein the MMP-9 inhibitor is selected from the group consisting of doxycycline, minocycline, pravastatin, captopril and a beta blocker.

24. The composition of claim 20, wherein the ROCK inhibitor is belumosudil and the MMP-9 inhibitor is minocycline.
